(19) United States
(12) Patent Application Publication
(10) Pub. No.: US 2025/0114741 A1
(43) Pub. Date: Apr. 10, 2025

(54) POWER RECOVERY DURING GAS SEPARATION

(71) Applicant: SABIC GLOBAL TECHNOLOGIES B.V., Bergen op Zoom (NL)

(72) Inventors: Israr Ul Haque, Houston, TX (US); Ali Essa Al Hammad, Riyadh (SA); Muammar Mohammad Al-Ansari, Riyadh (SA); Awais Ahmed, Riyadh (SA); Ahmed Saeed Al-Khalaf, Riyadh (SA)

(21) Appl. No.: 18/721,501

(22) PCT Filed: Dec. 19, 2022
(86) PCT No.: PCT/IB2022/062493

§ 371 Date: Jun. 18, 2024

(30) Foreign Application Priority Data
Dec. 23, 2021 (EP) 21217235.7

(51) Int. Cl. B01D 53/22; B01D 53/047; C01C 1/04; C07C 29/76

(52) U.S. Cl. CPC B01D 53/229; B01D 53/047; C01C 1/04; C07C 29/76; B01D 2256/16; B01D 2257/102; B01D 2257/502; B01D 2257/504; B01D 2257/7025; B01D 2257/80

(57) ABSTRACT

A method for recovering power during gas separation, the method including separating a mixed gas stream into a hydrogen-rich stream and a secondary stream; and expanding the secondary stream after the separating or expanding the mixed gas stream prior to the separating to recover power. The method may include using a membrane to separate the mixed gas stream into the hydrogen-rich stream and the secondary stream; and expanding the secondary stream.

### CROSS REFERENCE TO RELATED APPLICATION

This application claims priority to European Application 21217235.7, filed on Dec. 23, 2021, the content of which is incorporated by reference in its entirety.

### BACKGROUND

A chemical or molecule (e.g., monomer or polymer) synthesis assembly (e.g., a chemical plant) may include gas separation. For example, it may be desirable to separate hydrogen from a gas stream (also referred to herein as a “purge stream”) in a chemical or molecule synthesis assembly for further use in the chemical or molecule synthesis assembly.

### BRIEF DESCRIPTION

As described above, conventional practice provides for separation of hydrogen from a purge stream in a chemical or molecule synthesis assembly for further use in the chemical or molecule synthesis assembly. Opportunities for reducing costs associated with operating a chemical or molecule synthesis assembly, for example, costs associated with supplying power for use in the chemical or molecule synthesis assembly, exist and are addressed by the systems, processes, and methods of the present disclosure. A solution to reduce costs associated with conventional operation of a chemical or molecule synthesis assembly has been discovered. Accordingly, disclosed, in various embodiments, are power recovery systems, methods for recovering power during gas separation, and methods of methanol or ammonia synthesis.

Disclosed herein is a method for recovering power during gas separation, the method including separating a mixed gas stream into a hydrogen-rich stream and a secondary stream; and expanding the secondary stream after the separating or expanding the mixed gas stream prior to the separating to recover power.

Disclosed herein is a power recovery system including a gas separator configured to separate a mixed gas stream into a hydrogen-rich stream and a secondary stream; an expander configured to expand the mixed gas stream prior to being separated, or the secondary stream after being separated; and a compressor configured to compress the hydrogen-rich stream using power recovered from expansion of the mixed gas stream or expansion of the secondary stream.

Disclosed herein is a method of methanol or ammonia synthesis, the method including forming methanol or ammonia in a methanol or ammonia synthesis assembly; withdrawing a mixed gas stream from the methanol or ammonia synthesis assembly; separating the mixed gas stream into a hydrogen-rich stream and a secondary stream; expanding the secondary stream after the separating or expanding the mixed gas stream prior to the separating to recover power; compressing the hydrogen-rich stream using at least a portion of the power recovered from expansion of the secondary stream or expansion of the mixed gas stream to provide a compressed hydrogen-rich stream; and recycling the compressed hydrogen-rich stream to the methanol or ammonia synthesis assembly.

The above described and other features are exemplified by the following figures and detailed description.

Any combination or permutation of embodiments is envisioned. Additional advantageous features, functions and applications of the disclosed systems, processes, and methods of the present disclosure will be apparent from the description which follows, particularly when read in conjunction with the appended figures.

### BRIEF DESCRIPTION OF THE DRAWINGS

The following figures are exemplary embodiments wherein the like elements are numbered alike.

Exemplary embodiments of the present disclosure are further described with reference to the appended figures. It is to be noted that the various features, steps, and combinations of features/steps described below can be arranged and organized differently to result in embodiments which are still within the scope of the present disclosure. To assist those of ordinary skill in the art in making, using, and practicing the disclosed systems, methods, and processes, reference is made to the appended figures, wherein:

FIG. 1 is a diagram of an embodiment including a membrane.

FIG. 2 is a diagram of an embodiment including a membrane.

FIG. 3 is a diagram of an embodiment including a pressure swing adsorber.

### DETAILED DESCRIPTION

The exemplary embodiments disclosed herein are illustrative of advantageous power recovery systems, methods for recovering power during gas separation, and methods of methanol or ammonia synthesis. It should be understood, however, that the disclosed embodiments are merely exemplary of the present disclosure, which may be embodied in various forms. Therefore, details disclosed herein with reference to exemplary systems, methods, and processes are not to be interpreted as limiting, but merely as the basis for teaching one skilled in the art the advantageous systems, methods, and processes of the present disclosure.

The present inventors have found that power can be recovered during gas separation of a mixed gas stream into a hydrogen-rich stream and a secondary stream. As used herein, the term “hydrogen-rich” refers to a gas stream that includes 70 to 100 mole percent (mol %) hydrogen, for example, 80 to 98 mol % hydrogen or 90 to 95 mol % hydrogen, based on a total number of moles of the purge gas stream. The hydrogen-rich stream may also include methane, nitrogen, carbon dioxide, or a combination thereof. To recover power, the secondary stream may be expanded after the separating or the mixed gas stream may be expanded prior to the separating.

Examples of chemical or molecule synthesis assemblies in which it may be desirable to separate a mixed gas stream into a hydrogen-rich stream and a secondary stream include a methanol synthesis assembly, an ammonia synthesis assembly, or any process stream that can be separated, and one or both of the separated streams can be expanded to recover power that can be used to pressurize the other stream, or the recovered power can be used for any other use or purpose, or a combination thereof.

Methanol synthesis assemblies may include sections such as frontend (in which syngas (a mixture of H₂, CO₂, CO and CH₄) is generated), synthesis loop (in which methanol is synthesized using syngas at relatively high pressure), and distillation (in which raw methanol is purified, and potentially prepared for sale). Methanol molecules contains both carbon and hydrogen, and the synthesis loop may use about 70 mol % of the hydrogen produced in the frontend, and a part of the remaining 30 mol % of hydrogen produced in the frontend, along with undesired inert gases (such as methane and nitrogen) may be purged. The purge stream including 30 mol % of hydrogen produced in the frontend may be separated into a secondary stream and a hydrogen-rich stream that may be used for further methanol synthesis. The secondary stream may be used for fuel. Prior to being used for fuel, the secondary stream may be depressurized.

In an embodiment, pressure energy may be recovered in an expander, used to expand the secondary stream, coupled with an electric power generator, or the expander may be coupled directly to a compressor. The electric energy produced may be utilized to operate a compressor to pressurize the separated hydrogen-rich stream, or for another purpose within or outside of the synthesis assembly. In an embodiment, no additional or external energy is used to compress the separated hydrogen-rich stream prior to being used for further methanol synthesis. In an embodiment, the power recovered from the expander may also be supplemented with power provided by a helper motor, a steam turbine, or a combination thereof to compress the separated hydrogen-rich stream.

In an embodiment, the separation may be performed using a membrane. When using a membrane, only a small portion, for example, 25 weight percent (wt %), of the total flow directed to the membrane may be permeated and recovered as a hydrogen-rich (permeate) stream. The pressure of the stream fed to the membrane may be, for example, 70 to 130 kg/cm², or 85 to 115 kg/cm² or 95 to 105 kg/cm², and the pressure of the hydrogen-rich (permeate) stream may be, for example, 5 to 55 kg/cm², or 15 to 45 kg/cm² or 25 to 35 kg/cm². The remaining 75 wt % of the total flow directed to the membrane, which is recovered as a retentate stream, exits the membrane with only a small pressure drop. The retentate stream may be used for fuel, with a pressure of not more than 5 kg/cm², and thus, may be depressurized prior to being used for fuel. In an embodiment, the pressure of the retentate stream may be used to pressurize the hydrogen-rich (permeate) stream.

Membrane processes may use the pressure differential of the stream being separated, and the pressure of the hydrogen-rich (permeate) stream may be significantly reduced as compared to the stream being separated. It may be desirable to compress the hydrogen-rich (permeate) stream prior to further use, for example, in methanol synthesis. Pressurizing a hydrogen-rich (permeate) stream may be an energy intensive process, involving use of significant power.

Accordingly, the method may include using a membrane to separate the mixed gas stream into the hydrogen-rich stream and the secondary stream; and expanding the secondary stream, and the secondary stream may include a retentate stream. The method may further include heating the secondary stream prior to expanding the secondary stream. The method may further include heating the mixed gas stream prior to separating the mixed gas stream. The method may further include compressing the hydrogen-rich stream using at least a portion of the power recovered from expansion of the secondary stream or expansion of the mixed gas stream to provide a compressed hydrogen-rich stream.

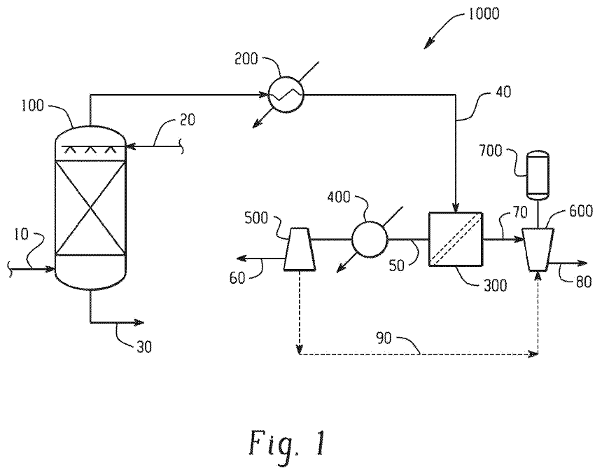

For example, as shown in FIG. 1, stream 10 in a portion of a methanol synthesis assembly 1000 may be washed with water 20 in a methanol wash column 100 to provide a bottoms stream 30 that may be fed to a distillation column and a stream 40 that is fed to membrane 300. Stream 40 exiting methanol wash column 100 may optionally be heated in heat exchanger 200 prior to being fed to membrane 300. Membrane 300 separates stream 40 into retentate stream 50 and hydrogen-rich (permeate) stream 70. Retentate stream 50 exiting membrane 300 may optionally be heated in heat exchanger 400 prior to being fed to expander 500, which forms expanded retentate stream 60, which may be used for fuel. Hydrogen-rich (permeate) stream 70 exiting membrane 300 is fed to compressor 600, which forms compressed hydrogen-rich (permeate) stream 80. Power 90 recovered from expander 500 is used to power compressor 600, which may optionally include helper motor 700. Surplus power 90 that is not used to power compressor 600 may be separately recovered. Expander 500 and compressor 600 may be mechanically coupled, and in an aspect may be mounted on a single shaft. Other mechanical, electrical, and/or hydraulic methods of power transfer between expander 500 and compressor 600 may be used.

Compressed hydrogen-rich (permeate) stream 80 may be fed to the synthesis loop, a syngas regeneration section, or a combination thereof. Prior to being fed to the synthesis loop, the syngas regeneration section, or the combination thereof, the compressed hydrogen-rich (permeate) stream 80 may be pressurized to a pressure of the synthesis loop, the syngas regeneration section, or the combination thereof using the compressor 600.

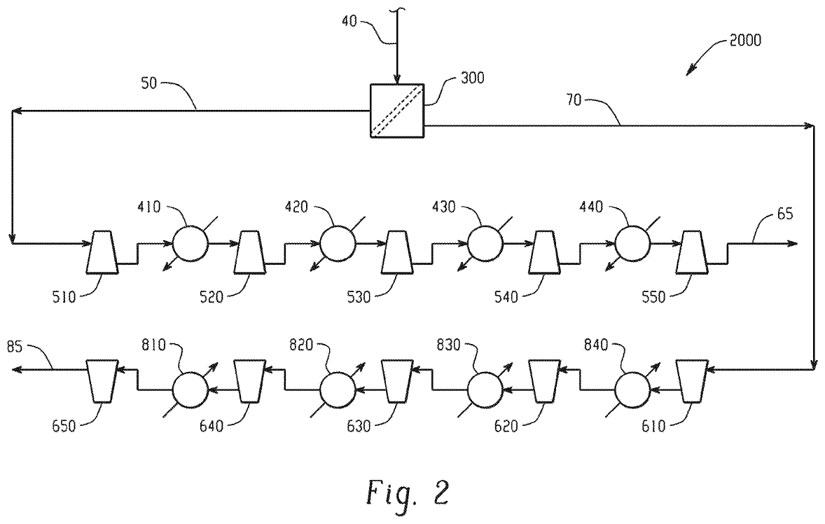

As shown in FIG. 2, in an expander-compressor assembly 2000, membrane 300 separates stream 40 into retentate stream 50 and hydrogen-rich (permeate) stream 70. The pressure of retentate stream 50 may be expanded in stages in a series of expanders 510, 520, 530, 540, 550, which form expanded retentate stream 65, and the power recovered may be used to pressurize hydrogen-rich (permeate) stream 70 in a series of compressors 610, 620, 630, 640, 650, which form compressed hydrogen-rich (permeate) stream 85. In an embodiment, heat exchangers 410, 420, 430, 440 may be disposed between expanders 510, 520, 530, 540, 550, respectively, and heat exchangers 810, 820, 830, 840 may be disposed between compressors 610, 620, 630, 640, 650, respectively.

The pressure of retentate stream 50 entering expander 510 may be in a range of, for example, 70 to 130 kg/cm², or 85 to 115 kg/cm² or 95 to 105 kg/cm². The pressure of hydrogen-rich (permeate) stream 70 entering compressor 610 may be in a range of, for example, 40 to 90 kg/cm², or 50 to 80 kg/cm² or 60 to 70 kg/cm². The pressure of expanded retentate stream 65 exiting expander 550 may be in a range of, for example, 1 to 30 kg/cm², or 2 to 20 kg/cm² or 3 to 10 kg/cm². The pressure of compressed hydrogen-rich (permeate) stream 85 exiting compressor 650 may be in a range of, for example, 70 to 130 kg/cm², or 85 to 115 kg/cm² or 95 to 105 kg/cm².

The synthesis loop of a methanol synthesis assembly may operate at 100 barg (0.1 megapascal), and it may not be advisable to install a pressure swing adsorber on such a relatively high pressure system (for example, it may not be advisable to feed a purge stream of the assembly to a pressure swing adsorber). The purge stream pressure may be reduced by installing an expander upstream of a pressure swing adsorber. The hydrogen-rich stream separated in the pressure swing adsorber, which may operate at lower pressure than the synthesis loop, may then be pressurized to a pressure of the synthesis loop by recovering power from the expander.

The method may include expanding the mixed gas stream prior to separating the mixed gas stream into the hydrogen-rich stream and the secondary stream to provide an expanded mixed gas stream; and using pressure swing adsorption to separate the expanded mixed gas stream into the hydrogen-rich stream and the secondary stream, and the secondary stream may include a tail gas stream. The method may further include heating the mixed gas stream prior to expanding the mixed gas stream.

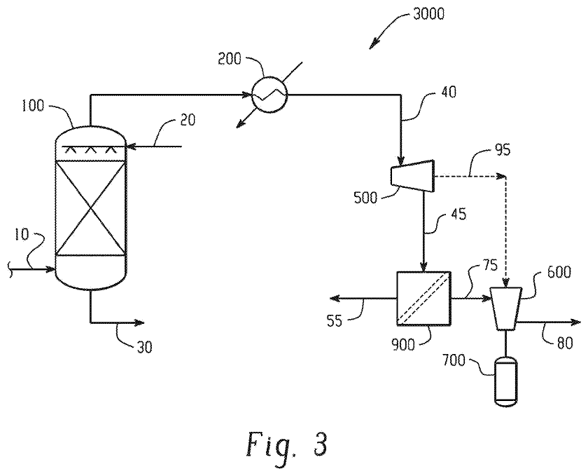

For example, as shown in FIG. 3, stream 10 in a portion of a methanol synthesis assembly 3000 may be washed with water 20 in a methanol wash column 100 to provide a bottoms stream 30 that may be fed to a distillation column and a stream 40 that may be heated or cooled by heat exchanger 200, and then fed to expander 500, thereby forming an expanded stream 45. Pressure swing adsorber 900 separates expanded stream 45 into tail gas 55, which may be used for fuel, and hydrogen-rich stream 75. Hydrogen-rich stream 75 exiting pressure swing adsorber 900 is fed to compressor 600, which forms compressed hydrogen-rich stream 80. Power 95 recovered from expander 500 is used to power compressor 600, which may optionally include helper motor 700. Power recovered from expander 500 that is not used to power compressor 600 may be separately recovered. Compressed hydrogen-rich stream 80 may be fed to the synthesis loop. Expander 500 and compressor 600 may be mechanically coupled, and in an aspect may be mounted on a single shaft. Other mechanical, electrical, and/or hydraulic methods of power transfer between expander 500 and compressor 600 may be used.

In an embodiment, a power recovery system includes a gas separator configured to separate a mixed gas stream into a hydrogen-rich stream and a secondary stream; an expander configured to expand the mixed gas stream prior to being separated, or the secondary stream after being separated; and a compressor configured to compress the hydrogen-rich stream using power recovered from expansion of the mixed gas stream or expansion of the secondary stream.

The gas separator may include a membrane; the secondary stream may include a retentate stream; and the expander is configured to expand the retentate stream. The system may further include a heater configured to heat the retentate stream downstream of the membrane and upstream of the expander. The system may further include a heater configured to heat the mixed gas stream upstream of the membrane.

The gas separator may include a pressure swing adsorber; the secondary stream may include a tail gas stream; and the expander is configured to expand the mixed gas stream. The system may further include a heater configured to heat the mixed gas stream upstream of the expander.

In an embodiment, a method of methanol or ammonia synthesis includes forming methanol or ammonia in a methanol or ammonia synthesis assembly; withdrawing a mixed gas stream from the methanol or ammonia synthesis assembly; separating the mixed gas stream into a hydrogen-rich stream and a secondary stream; expanding the secondary stream after the separating or expanding the mixed gas stream prior to the separating to recover power; compressing the hydrogen-rich stream using at least a portion of the power recovered from expansion of the secondary stream or expansion of the mixed gas stream to provide a compressed hydrogen-rich stream; and recycling the compressed hydrogen-rich stream to the methanol or ammonia synthesis assembly.

The method may include using a membrane to separate the mixed gas stream into the hydrogen-rich stream and the secondary stream, the secondary stream including a retentate stream, and expanding the retentate stream. The method may include expanding the mixed gas stream prior to separating the mixed gas stream into the hydrogen-rich stream and the secondary stream to provide an expanded mixed gas stream, and using pressure swing adsorption to separate the expanded mixed gas stream into the hydrogen-rich stream and the secondary stream, the secondary stream including a tail gas stream.

A membrane may be used, for example, in a methanol synthesis assembly, not only to separate a hydrogen-rich stream from a purge stream of the assembly, but also to reduce accumulation of inert gases, for example, nitrogen, methane, or a combination thereof, in the synthesis loop. For example, a membrane may be used to separate nitrogen (in a retentate stream) from a hydrogen-rich (permeate) stream, which may be added to the synthesis loop. Addition of nitrogen to the synthesis loop may cause undesirable accumulation of inert gasses in the synthesis loop, and limit per pass conversion in a methanol converter of the synthesis loop. Separation of nitrogen and other inert gases from the hydrogen-rich stream fed to the synthesis loop may improve methanol converter performance.

While reference is made herein to a methanol synthesis assembly, the disclosed systems, processes, and methods are similarly applicable to an ammonia synthesis assembly, which may similarly be operated at a relatively high pressure. Similar to the methanol synthesis assembly, a purge stream may be removed from the synthesis loop of an ammonia synthesis assembly to maintain a desired concentration of inert gases in the synthesis loop for improved per pass conversion. Hydrogen separated from the purge stream may be further used in the ammonia synthesis assembly.

Use of a membrane to reduce accumulation of inert gases may be more effective when an external carbon source, for example, CO₂, is added upstream of the synthesis loop. Use of a membrane to reduce accumulation of inert gases may also be more effective if the frontend uses a hydrogen production technology, for example, autothermal reforming (ATR), a gasifier, partial oxidation (POX), or a combination thereof, with a relatively low ratio of hydrogen to carbon oxide in the synthesis gas. Use of a membrane, with CO₂ addition, may also reduce synthesis assembly energy by reducing use of the natural gas, reducing the fuel usage in a primary reformer, or a combination thereof.

This disclosure is further illustrated by the following example, which is non-limiting.

### Example

A process model was simulated using Aspen Plus® V10 software. A feed stream 40 fed to a membrane was separated into a retentate stream 50 and a permeate stream 70. The composition of Streams 40, 50, 70 are provided in Table 1.

TABLE 1

| | Membrane | Retentate | Permeate |
|---|---|---|---|
| Component (Mole percent) | Feed 40 | Stream 50 | Stream 70 |
| H₂ | 78.8 | 53.9 | 93.2 |
| N₂ | 8.9 | 19.7 | 2.6 |
| CH₄ | 10.2 | 23.6 | 2.5 |
| CO | 0.8 | 1.6 | 0.3 |
| CO₂ | 1.2 | 1.2 | 1.2 |
| H₂O | 0.1 | 0 | 0.2 |
| Methanol | 0 | 0 | 0 |
| Parameter | | | |
| Mole Flow (kilomoles per hour | 4479 | 1638 | 2841 |
| (kmole/hr)) | | | |
| Mass Flow (kilograms per hour | 29022 | 18629 | 10393 |
| (kg/hr)) | | | |
| Temperature (° C.) | 60 | 54 | 60 |
| Pressure (kilograms per square | 100 | 99.5 | 65 |
| centimeter (kg/cm²), Gauge (G) | | | |

The results of Table 1 show that the membrane can effectively produce a hydrogen-rich (permeate) stream and a retentate stream. The hydrogen-rich (permeate) stream is provided at a relatively high pressure of 99.5 kg/cm² compared to the pressure of the retentate stream of 65 kg/cm².

This disclosure further encompasses the following aspects.

Aspect 1. A method for recovering power during gas separation, the method comprising: separating a mixed gas stream into a hydrogen-rich stream and a secondary stream; and expanding the secondary stream after the separating or expanding the mixed gas stream prior to the separating to recover power.

Aspect 2. The method of Aspect 1, comprising: using a membrane to separate the mixed gas stream into the hydrogen-rich stream and the secondary stream; and expanding the secondary stream; wherein the secondary stream comprises a retentate stream.

Aspect 3. The method of Aspect 2, further comprising heating the secondary stream prior to expanding the secondary stream.

Aspect 4. The method of any of Aspects 2 or 3, further comprising heating the mixed gas stream prior to separating the mixed gas stream.

Aspect 5. The method of Aspect 1, comprising: expanding the mixed gas stream prior to separating the mixed gas stream into the hydrogen-rich stream and the secondary stream to provide an expanded mixed gas stream; and using pressure swing adsorption to separate the expanded mixed gas stream into the hydrogen-rich stream and the secondary stream; wherein the secondary stream comprises a tail gas stream.

Aspect 6. The method of Aspect 5, further comprising heating the mixed gas stream prior to expanding the mixed gas stream.

Aspect 7. The method of any of the preceding aspects, further comprising compressing the hydrogen-rich stream using at least a portion of the power recovered from expansion of the secondary stream or expansion of the mixed gas stream to provide a compressed hydrogen-rich stream.

Aspect 8. A power recovery system comprising: a gas separator configured to separate a mixed gas stream into a hydrogen-rich stream and a secondary stream; an expander configured to expand the mixed gas stream prior to being separated, or the secondary stream after being separated; and a compressor configured to compress the hydrogen-rich stream using power recovered from expansion of the mixed gas stream or expansion of the secondary stream.

Aspect 9. The system of Aspect 8, wherein the gas separator comprises a membrane; the secondary stream comprises a retentate stream; and the expander is configured to expand the retentate stream.

Aspect 10. The system of Aspect 9, further comprising a heater configured to heat the retentate stream downstream of the membrane and upstream of the expander.

Aspect 11. The system of Aspect 9 or 10, further comprising a heater configured to heat the mixed gas stream upstream of the membrane.

Aspect 12. The system of Aspect 8, wherein the gas separator comprises a pressure swing adsorber; the secondary stream comprises a tail gas stream; and the expander is configured to expand the mixed gas stream.

Aspect 13. The system of Aspect 12, further comprising a heater configured to heat the mixed gas stream upstream of the expander.

Aspect 14. A method of methanol or ammonia synthesis, the method comprising: forming methanol or ammonia in a methanol or ammonia synthesis assembly; withdrawing a mixed gas stream from the methanol or ammonia synthesis assembly; separating the mixed gas stream into a hydrogen-rich stream and a secondary stream; expanding the secondary stream after the separating or expanding the mixed gas stream prior to the separating to recover power; compressing the hydrogen-rich stream using at least a portion of the power recovered from expansion of the secondary stream or expansion of the mixed gas stream to provide a compressed hydrogen-rich stream; and recycling the compressed hydrogen-rich stream to the methanol or ammonia synthesis assembly.

Aspect 15. The method of Aspect 14, comprising: using a membrane to separate the mixed gas stream into the hydrogen-rich stream and the secondary stream, the secondary stream comprising a retentate stream, and expanding the retentate stream; or expanding the mixed gas stream prior to separating the mixed gas stream into the hydrogen-rich stream and the secondary stream to provide an expanded mixed gas stream, and using pressure swing adsorption to separate the expanded mixed gas stream into the hydrogen-rich stream and the secondary stream, the secondary stream comprising a tail gas stream.

The systems, methods, and processes can alternatively comprise, consist of, or consist essentially of, any appropriate materials, steps, or components herein disclosed. The systems, methods, and processes can additionally, or alternatively, be formulated so as to be devoid, or substantially free, of any materials (or species), steps, or components, that are otherwise not necessary to the achievement of the function or objectives of the systems, methods, and processes.

All ranges disclosed herein are inclusive of the endpoints, and the endpoints are independently combinable with each other (e.g., ranges of “up to 25 wt %, or, more specifically, 5 wt % to 20 wt %”, are inclusive of the endpoints and all intermediate values of the ranges of “5 wt % to 25 wt %,” etc.). “Combinations” is inclusive of blends, mixtures, alloys, reaction products, and the like. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless specified to the contrary herein, all test standards are the most recent standards in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

Although the processes and methods of the present disclosure have been described with reference to exemplary embodiments thereof, the present disclosure is not limited to such exemplary embodiments and/or implementations. Rather, the processes and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

1. A method for recovering power during gas separation, the method comprising:
separating a mixed gas stream into a hydrogen-rich stream and a secondary stream; and
expanding the secondary stream after the separating or expanding the mixed gas stream prior to the separating to recover power.

2. The method of claim 1, comprising:
using a membrane to separate the mixed gas stream into the hydrogen-rich stream and the secondary stream; and
expanding the secondary stream;
wherein the secondary stream comprises a retentate stream.

3. The method of claim 2, further comprising heating the secondary stream prior to expanding the secondary stream.

4. The method of any of claim 2, further comprising heating the mixed gas stream prior to separating the mixed gas stream.

5. The method of claim 1, comprising:
expanding the mixed gas stream prior to separating the mixed gas stream into the hydrogen-rich stream and the secondary stream to provide an expanded mixed gas stream; and
using pressure swing adsorption to separate the expanded mixed gas stream into the hydrogen-rich stream and the secondary stream;
wherein the secondary stream comprises a tail gas stream.

6. The method of claim 5, further comprising heating the mixed gas stream prior to expanding the mixed gas stream.

7. The method of claim 1, further comprising compressing the hydrogen-rich stream using at least a portion of the power recovered from expansion of the secondary stream or expansion of the mixed gas stream to provide a compressed hydrogen-rich stream.

8. A power recovery system comprising:
a gas separator configured to separate a mixed gas stream into a hydrogen-rich stream and a secondary stream;
an expander configured to expand
the mixed gas stream prior to being separated, or
the secondary stream after being separated; and
a compressor configured to compress the hydrogen-rich stream using power recovered from expansion of the mixed gas stream or expansion of the secondary stream.

9. The system of claim 8, wherein
the gas separator comprises a membrane;
the secondary stream comprises a retentate stream; and
the expander is configured to expand the retentate stream.

10. The system of claim 9, further comprising a heater configured to heat the retentate stream downstream of the membrane and upstream of the expander.

11. The system of claim 9, further comprising a heater configured to heat the mixed gas stream upstream of the membrane.

12. The system of claim 8, wherein
the gas separator comprises a pressure swing adsorber;
the secondary stream comprises a tail gas stream; and
the expander is configured to expand the mixed gas stream.

13. The system of claim 12, further comprising a heater configured to heat the mixed gas stream upstream of the expander.

14. A method of methanol or ammonia synthesis, the method comprising:
forming methanol or ammonia in a methanol or ammonia synthesis assembly;
withdrawing a mixed gas stream from the methanol or ammonia synthesis assembly;
separating the mixed gas stream into a hydrogen-rich stream and a secondary stream;
expanding the secondary stream after the separating or expanding the mixed gas stream prior to the separating to recover power;
compressing the hydrogen-rich stream using at least a portion of the power recovered from expansion of the secondary stream or expansion of the mixed gas stream to provide a compressed hydrogen-rich stream; and
recycling the compressed hydrogen-rich stream to the methanol or ammonia synthesis assembly.

15. The method of claim 14, comprising:
using a membrane to separate the mixed gas stream into the hydrogen-rich stream and the secondary stream, the secondary stream comprising a retentate stream, and expanding the retentate stream; or
expanding the mixed gas stream prior to separating the mixed gas stream into the hydrogen-rich stream and the secondary stream to provide an expanded mixed gas stream, and using pressure swing adsorption to separate the expanded mixed gas stream into the hydrogen-rich stream and the secondary stream, the secondary stream comprising a tail gas stream.
